(19) United States
(12) Patent Application Publication
(10) Pub. No.: US 2006/0122230 A1
(43) Pub. Date: Jun. 8, 2006

(54) 1,5-Diaryl-pyrrole-3-carboxamide derivatives and their use as cannabinoid receptor modulators

(75) Inventors: Anna Ingrid Kristina Berggren, Molndal (SE); Stig Jonas Bosfrom, Molndal (SE); Leifeng Cheng, Molndal (SE); Stig Thomas Elebring, Molndal (SE); Peter Greasley, Molndal (SE); Mats Nagard, Molndal (SE); Johan Michael Wilstermann, Molndal (SE); Emma Terricabras, Barcelona (ES)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/540,276

(22) PCT Filed: Dec. 18, 2003
(86) PCT No.: PCT/GB03/05569

§ 371 Date: Jun. 21, 2005

(30) Foreign Application Priority Data
Dec. 24, 2002 (GB) 0230088.7

(51) Int. Cl. A61K 31/454; A61K 31/4015; A61K 31/401

(52) U.S. Cl. 514/326; 514/423; 514/424; 546/207; 548/537

(57) ABSTRACT

The present invention relates to a compound of formula (I) in which R¹ and R² independently represent phenyl, thienyl or pyridyl each of which is optionally substituted by one, two or three groups represented by Z; and R³ is H, a C₁₋₃alkyl group, a C₁₋₃alkoxymethyl group, trifluoromethyl, a hydroxyC₁₋₃alkyl group, an aminoC₁₋₃alkyl group, C₁₋₃alkoxycarbonyl, carboxy, cyano, carbamoyl, mono or di C₁₋₃alkylcarbamoyl, acetyl, or hydrazinocarbonyl of formula —CONHNR^{a}R^{b} wherein R^{a} and R^{b} are as defined for R⁴ and R⁵ respectively; X is CO or SO₂; Y is absent or represents NH optionally substituted by a C₁₋₃alkyl group; R⁴ and R⁵ independently represent: a C₁₋₆alkyl group; an (amino)C₁₋₄alkyl-group in which the amino is optionally substituted by one or more C₁₋₃alkyl groups; an optionally substituted non-aromatic C₃₋₁₅carbocyclic group; a (C₃₋₁₂cycloalkyl)C₁₋₃alkyl-group; a group —(CH₂)ᵣ(phenyl)ₛ; naphthyl; anthracenyl; a saturated 5 to 8 membered heterocyclic group containing one nitrogen and optionally one of the following: oxygen, sulphur or an additional nitrogen wherein the heterocyclic group is optionally substituted; 1-adamantylmethyl; a group —(CH₂)ₜ Het where Het represents an aromatic heterocycle optionally substituted; or R⁴ represents H and R⁵ is as defined above; or R⁴ and R⁵ together with the nitrogen atom to which they are attached represent a saturated 5 to 8 membered heterocyclic group; R⁶ is H, a C₁₋₃alkyl group, a C₁₋₃alkoxymethyl group, trifluoromethyl, a hydroxyC₁₋₃alkyl group, C₁₋₃alkoxycarbonyl, carboxy, cyano, carbamoyl, mono or di C₁₋₃alkylcarbamoyl, acetyl, or hydrazinocarbonyl of formula —CONHNR^{a}R^{b}; with provisos; to processes for preparing such compounds, to their use in the treatment of obesity, psychiatric and neurological disorders particularly obesity, to methods for their therapeutic use and to pharmaceutical compositions containing them.

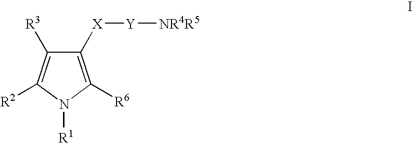

### FIELD OF INVENTION

The present invention relates to certain pyrrole carboxamide compounds of formula I, to processes for preparing such compounds, to their use in the treatment of obesity, psychiatric and neurological disorders, and to pharmaceutical compositions containing them.

### BACKGROUND OF THE INVENTION

It is known that certain CB₁ modulators (known as antagonists or inverse agonists) are useful in the treatment of obesity, psychiatric and neurological disorders (WO01/70700 and EP 656354). However, there is a need for CB₁ modulators with improved physicochemical properties and/or DMPK properties and/or pharmacodynamic properties.

1,5-Diarylpyrrole-3-carboxamides are reported to have antifungal activity in Il Farmaco 1988, vol XLIII, N9 665, M. Scalzo et al, Il Farmaco 1988, vol 43, N9 677, M. Scalzo et al, Il Farmaco 1989, vol 44, N1 65, C. G. Porretta et al, and Eur. J Med. Chem 1992, 27, 701 F Cerretto et al. All compounds disclosed in these documents are disclaimed from the compound claims of the present application.

U.S. Pat. No. 6,248,894 discloses certain pyrroles have anti-fungal activity. All compounds disclosed in this document are disclaimed from the compound claims of the present application.

WO01/58869 discloses that certain 1-(2-morpholinoethyl)pyrrolecarboxamides are useful in treating respiratory diseases.

### DESCRIPTION OF THE INVENTION

The invention relates to a compound of formula (I) 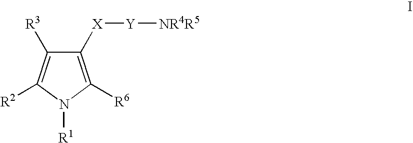
and pharmaceutically acceptable salts, prodrugs, solvates and crystalline forms thereof, in which
R¹ and R² independently represent phenyl, thienyl or pyridyl each of which is optionally substituted by one, two or three groups represented by Z;
Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, mono or di C₁₋₃alkylaimdo, C₁₋₃alkylsulphonyl, C₁₋₃alkoxycarbonyl, carboxy, cyano, carbamoyl, mono or di C₁₋₃alkyl carbamoyl, sulphamoyl and acetyl; and
R³ is H, a C₁₋₃alkyl group, a C₁₋₃alkoxymethyl group, trifluoromethyl, a hydroxyC₁₋₃alkyl group, an aminoC₁₋₃alkyl group, C₁₋₃akoxycarbonyl, carboxy, cyano, carbamoyl, mono or di C₁₋₃alkylcarbamoyl, acetyl, or hydrazinocarbonyl of formula —CONHNR^{a}R^{b} wherein R^{a} and R^{b} are as defined for R⁴ and R⁵ respectively and;
X is CO or SO₂;
Y is absent or represents NH optionally substituted by a C₁₋₃alkyl group;
R⁴ and R⁵ independently represent:
a C₁₋₆alkyl group;
an (amino)C₁₋₄alkyl-group in which the amino is optionally substituted by one or more C₁₋₃alkyl groups;
an optionally substituted non-aromatic C₃₋₁₅carbocyclic group;
a (C₃₋₁₂cycloalkyl)C₁₋₃alkyl-group;
a group —(CH₂)ᵣ(phenyl )ₛ in which r is 0,1, 2, 3 or 4, s is 1 when r is 0 otherwise s is 1 or 2 and the phenyl groups are optionally independently substituted by one, two or three groups represented by Z;
naphthyl;
anthracenyl;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
1-adamantylmethyl;
a group —(CH₂)ₜ Het in which t is 0,1, 2, 3 or 4, and the alkylene chain is optionally substituted by one or more C₁₋₃alkyl groups and Het represents an aromatic heterocycle optionally substituted by one, two or three groups selected from a C₁₋₅alkyl group, a C₁₋₅alkoxy group or halo;
or R⁴ represents H and R⁵ is as defined above;
or R⁴ and R⁵ together with the nitrogen atom to which they are attached represent a saturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy or benzyl;
R⁶ is H, a C₁₋₃alkyl group, a C₁₋₃alkoxymethyl group, trifluoromethyl, a hydroxyC₁₋₃alkyl group, an aminoC₁₋₃alkyl group, C₁₋₃alkoxycarbonyl, carboxy, cyano, carbamoyl, mono or di C₁₋₃alkylcarbamoyl, acetyl, or hydrazinocarbonyl of formula —CONHNR^{a}R^{b} wherein R^{a} and R^{b} are as defined for R⁴ and R⁵ respectively and;
with the proviso that when R⁶ is methyl then the group X—Y—NR⁴R⁵ does not represent CONHC₆H₁₃, CONHC₁₂H₂₅, CONH₂, CONHCH₃, CON(CH₃)₂, 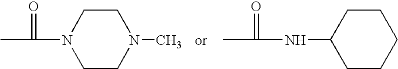
   and with the further proviso that when R¹ and R² independently represent phenyl then Z is not an ortho methyl group.

In a particular group of compounds of formula I Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, amino, mono or di C₁₋₃alkylamino, mono or di C₁₋₃alkylamido, C₁₋₃alkylsulphonyl, C₁₋₃alkoxycarbonyl, carboxy, cyano, carbamoyl, mono or di C₁₋₃alkyl carbamoyl, sulphamoyl and acetyl.

Further values of R¹, R², R³ , X—Y—NR⁴R⁵ and R⁶ in compounds of formula I now follow. It will be understood that such values may be used where appropriate with any of the definitions, claims or embodiments defined hereinbefore or hereinafter.

In one group of compounds of formula I, R¹ represents phenyl optionally substituted by halo or C₁₋₃alkoxy located in the 2 and 4 positions of the phenyl ring. In such compounds R¹ is selected from phenyl, 4-chlorophenyl, 2,4-dichlorophenyl and 4-methoxyphenyl.

In a second group of compounds of formula I, R² represents phenyl optionally substituted by halo or C₁₋₃alkoxy located in the 2 and 4 positions of the phenyl ring. In such compounds R¹ is selected from phenyl, 2,4-dichlorophenyl and 2,4-dimethoxyphenyl.

In a third group of compounds of formula I, X—Y—NR⁴R⁵ represents CONHPh or CONH(1-piperidyl).

In a fourth group of compounds of formula I, X—Y—NR⁴R⁵ represents CONH(1-piperidinyl).

In a fifth group of compounds of formula I, X—Y—NR⁴R⁵ represents CO(1-piperidinyl). In a sixth group of compounds of formula I, R⁶ represents methyl.

One group of compounds of the present invention relates to compounds of the general formula (II) 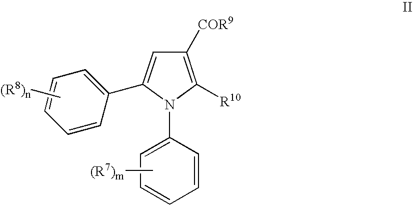
and pharmaceutically acceptable salts, prodrugs, and solvates in which
m represents 0,1, 2 or 3
R⁷ represents a C₁₋₆alkyl group, trifluoromethyl, a C₁₋₆alkoxy group, difluoromethoxy, trifluoromethoxy, or halo wherein when m is 2 or 3 then the groups R¹ may be the same or different;
n represents 0,1, 2 or 3;
R⁸ represents a C₁₋₆alkyl group, trifluoromethyl, a C₁₋₆alkoxy group, difluoromethoxy, trifluoromethoxy, or halo wherein when n is 2 or 3 then the groups R² may be the same or different;
R⁹ represents 1-piperidinyl, 1-piperidinylamino or anilino wherein the phenyl ring is optionally substituted by one or more of the following: a C₁₋₆alkyl group, trifluoromethyl, a C₁₋₆alkoxy group, difluoromethoxy, trifluoromethoxy or halo; and
R¹⁰ represents a C₁₋₆alkyl, C₁₋₆alkoxy, or a C₁₋₆alkylamino group;
with the proviso that the compound is not 1-{[1-(4-chlorophenyl)-5-phenyl-2-methyl-1H-pyrrol-3-yl]carbonyl}piperidine or 1-{[1-(2,4-dichlorophenyl)-5-phenyl-2-methyl-1H-pyrrol-3-yl]carbonyl}piperidine.

Further values of R⁷, R⁸, R⁹, R¹⁰ in compounds of formula I now follow. It will be understood that such values may be used where appropriate with any of the definitions, claims or embodiments defined hereinbefore or hereinafter.

In one group of compounds of formula II, m is 2 and the groups R⁷ are located in the 2 and 4 positions of the phenyl ring. In such compounds R⁷ is selected from chloro and methoxy and the groups R⁷ may be the same or different.

In a second group of compounds of formula II, n is 2 and the groups R⁸ are located in the 2 and 4 positions of the phenyl ring. In such compounds R⁸ is selected from chloro and methoxy and the groups R⁸ may be the same or different.

In a third group of compounds of formula II, R⁹ represents anilino.

In a fourth group of compounds of formula II, R⁹ represents 1-piperidinyl

In a fifth group of compounds of formula II, R⁹ represents 1-piperidinylamino.

In a sixth group of compounds of formula II, R¹⁰ represents methyl.

“Pharmaceutically acceptable salt”, where such salts are possible, include pharmaceutically acceptable acid addition salt. A suitable pharmaceutically acceptable salt of a compound of Formula I is, for example, an acid-addition salt of a compound of Formula I which is sufficiently basic, for example an acid-addition salt with an inorganic or organic acid such as hydrochloric, hydrobromic, sulphuric, trifluoroacetic, citric or maleic acid.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates. Isomers may be separated using conventional techniques, e.g. chromatography or fractional crystallisation The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent. All stereoisomers are included within the scope of the invention.

The following definitions shall apply throughout the specification and the appended claims.

Unless otherwise stated or indicated, the term “alkyl” denotes either a straight or branched alkyl group. Examples of said alkyl include methyl, ethyl, n-propyl, isopropyl, n-butyl, iso-butyl sec-butyl and t-butyl. Preferred alkyl groups are methyl, ethyl, propyl, isopropyl and tertiary butyl.

Unless otherwise stated or indicated, the term “alkoxy” denotes a group O-alkyl, wherein alkyl is as defined above.

Unless otherwise stated or indicated, the term “halo” shall mean fluorine, chlorine, bromine or iodine.

Specific compounds of the invention are:
2-methyl-N,1,5-triphenyl-1H-pyrrole-3-carboxamide;
1-(4-chlorophenyl)-2-methyl-N,5-diphenyl-1H-pyrrole-3-carboxamide;
1-(4-methoxyphenyl)-2-methyl-N,5-diphenyl-1H-pyrrole-3-carboxamide;
5-(2,4dichlorophenyl)-2-methyl-N,1-diphenyl-1H-pyrrole-3-carboxamide;
1-(4-chlorophenyl)-5-(2,4-dichlorophenyl)-2-methyl-N-phenyl-1H-pyrrole-3-carboxamide;
5-(2,4-dichlorophenyl)-1-(4-methoxyphenyl)-2-methyl-N-phenyl-1H-pyrrole-3-carboxamide;
5-(2,4-dimethoxyphenyl)-2-methyl-N,1-diphenyl-1H-pyrrole-3-carboxamide;
1-(4-chlorophenyl)-5-(2,4-dimethoxyphenyl)-2-methyl-N-phenyl-1H-pyrrole-3-carboxamide;
5-(2,4-dimethoxyphenyl)-1-(4-methoxyphenyl)-2-methyl-N-phenyl-1H-pyrrole-3-carboxamide;
2-methyl-1,5-diphenyl-N-piperidin-1-yl-1H-pyrrole-3-carboxamide;
1-(4chlorophenyl)-2-methyl-5-phenyl-N-piperidin-1-yl-1H-pyrrole-3-carboxamide;
1-(4-methoxyphenyl)-2-methyl-5-phenyl-N-piperidin-1-yl-1H-pyrrole-3-carboxamide;
5-(2,4-dichlorophenyl)-2-methyl-1-phenyl-N-piperidin-1-yl-1H-pyrrole-3-carboxamide;
1-(4-chlorophenyl)-5-(2,4-dichlorophenyl)-2-methyl-N-piperidin-1-yl-1H-pyrrole-3-carboxamide;
5-(2,4-dichlorophenyl)-1-(4-methoxyphenyl)-2-methyl-N-piperidin-1-yl-1H-pyrrole-3-carboxamide;
1-{[5-(2,4-dimethoxyphenyl)-2-methyl-1-phenyl-1H-pyrrol-3-yl]carbonyl}piperidine;
1-(4-chlorophenyl)-5-(2,4-dimethoxyphenyl)-2-methyl-N-piperidin-1-yl-1H-pyrrole-3-carboxamide; and
5-(2,4-dimethoxyphenyl)-1-(4methoxyphenyl)-2-methyl-N-piperidin-1-yl-1H-pyrrole-3-carboxamide;
1-[(2-methyl-1,5-diphenyl-1H-pyrrol-3-yl)carbonyl]piperidine;
1-{[1-(4methoxyphenyl)-2-methyl-5-phenyl-1H-pyrrol-3-yl]carbonyl}piperidine;
1-{[5-(2,4-dichlorophenyl)-2-methyl-1-phenyl-1H-pyrrol-3-yl]carbonyl}piperidine;
1-{[1-(4chlorophenyl)-5-(2,4dichlorophenyl)-2-methyl-1H-pyrrol-3-yl]carbonyl}piperidine;
1-{[5-(2,4-dichlorophenyl)-1-(4-methoxyphenyl)-2-methyl-1H-pyrrol-3-yl]carbonyl}piperidine;
1-{[1-(4-chlorophenyl)-5-(2,4-dimethoxyphenyl)-2-methyl-1H-pyrrol-3-yl]carbonyl}piperidine; and
1-{[5-(2,4-dimethoxyphenyl) -1-(4-methoxyphenyl)-2-methyl-1H-pyrrol-3-yl]carbonyl}piperidine;
and where applicable, optical isomers, tautomers, stereoisomers and racemates thereof as well as pharmaceutically acceptable salts, solvates and crystalline forms thereof.

It should be understood that the present invention includes each of the above compounds and any combination of two or more these compounds that is 2, 3, 4, 5, 6, 7, 8, 9, 10, 11, 12, 13, 14, 15, 16, 17, 18, 19, 20, 21, 22, 23, 24 or 25 of these compounds.

Methods of Preparation

The compounds of the invention may be prepared as outlined below according to any of the following methods. However, the invention is not limited to these methods, the compounds may also be prepared as described for structurally related compounds in the prior art. Compounds of formula I in which X is CO may be prepared by reacting a compound of formula III 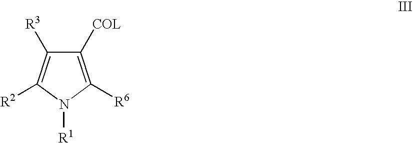
in which R¹, R², R³, and R⁶ are as previously defined and L represents hydroxy or halo e.g. chloro, with an amine of formula IV
R⁴R⁵YNH₂ IV
in which R⁴ and R⁵ are as previously defined in an inert solvent, for example dichloromethane, and optionally in the presence of a catalyst, for example a basic catalyst, eg 4-dimethylamino-pyridine, or optionally in the presence of a base for example triethylamine, at a temperature in the range of −25° C. to 150° C., and when L is hydroxy optionally in the presence of a coupling agent, for example a carbodiimide, eg 1-(3-dimethylaminopropyl)-3-ethylcarbodiimide.

Compounds of formula I in which X is SO₂ may be prepared by reacting a compound of formula V 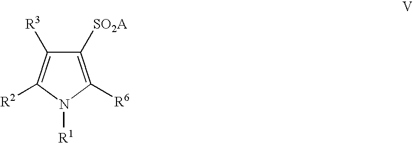
in which R¹, R², R³ and R⁶ are as previously defined and A represents halo with an amine of formula IV
R⁴R⁵NH₂ IV
in an inert solvent, for example dichloromethane, and optionally in the presence of a catalyst, for example a basic catalyst, eg 4-dimethylamino-pyridine, at a temperature in the range of −25° C. to 150° C.

Compounds of formula III may be prepared as described in the Examples and by other methods known to those skilled in the art. Certain compounds of formula III are novel and are claimed as a further aspect of the present invention as useful intermediates.

The compounds of the invention may be isolated from their reaction mixtures using conventional techniques.

Persons skilled in the art will appreciate that, in order to obtain compounds of the invention in an alternative and in some occasions, more convenient manner, the individual process steps mentioned hereinbefore may be performed in a different order, and/or the individual reactions may be performed at a different stage in the overall route (i.e. chemical transformations may be performed upon different intermediates to those associated hereinbefore with a particular reaction).

The expression “inert solvent” refers to a solvent which does not react with the starting materials, reagents, intermediates or products in a manner which adversely affects the yield of the desired product.

Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient either as a free base, or a pharmaceutically acceptable organic or inorganic acid addition salt, in a pharmaceutically acceptable dosage form Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight.

Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

A compound of the invention may also be combined with other anti-obesity agents such as Orlistat or a monoamine reuptake inhibitor, for example Sibutramine. Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatorheic hepatitis, osteoarthritis and some cancers) and psychiatric and neurological conditions.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

Pharmacological Properties

The compounds of formula (I) are useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders(e.g. Multiple Sclerosis), Raynaud's syndrome, Parkinson's disease, Huntington's chorea and Alzheimer's disease. The compounds are also potentially useful for the treatment of immune, cardiovascular, reproductive and endocrine disorders, septic shock and diseases related to the respiratory and gastrointestinal systems (e.g. diarrhea). The compounds are also potentially useful as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. The compounds may also eliminate the increase in weight which normally accompanies the cessation of smoking.

In another aspect the present invention provides a compound of formula I as claimed in any previous claim for use as a medicament.

In a further aspect the present invention provides the use of a compound of formula I including the compounds in the provisos in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms comprising administering a pharmacologically effective amount of a compound of formula I including the compounds in the provisos to a patient in need thereof.

The compounds of the present invention are particulary suitable for the treatment of obesity, e.g. by reduction of appetite and body weight, maintenance of weight reduction and prevention of rebound.

Combination Therapy

The compounds of the invention may be combined with another therapeutic agent that is useful in the treatment of disorders associated with the development and progress of obesity such as hypertension, hyperlipidaemias, dyslipidaemias, diabetes and atherosclerosis. For example, a compound of the present invention may be used in combination with a compound that affects thermogenesis, lipolysis, fat absorption, satiety, or gut motility. The compounds of the invention may be combined with another therapeutic agent that decreases the ratio of LDL:HDL or an agent that causes a decrease in circulating levels of LDL-cholesterol. In is patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration one or more of the following agents selected from:
a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-coagulant;
an omega-3 fatty acid;
another anti-obesity compound;
an antihypertensive compound for example an angiotensin converting enzyme (ACE) inhibitor, an angiotensin II receptor antagonist, an andrenergic blocker, an alpha andrenergic blocker, a beta andrenergic blocker, a mixed alpha/beta andrenergic blocker, an andrenergic stimulant, calcium channel blocker, an AT-1 blocker, a saluretic, a diuretic or a vasodilator;
a Melanin concentrating hormone (MCH) antagonist;
a PDK inhibitor; or
modulators of nuclear receptors for example LXR, FXR, RXR, and RORalpha;
an SSRI;
a serotonin antagonist;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Therefore in an additional feature of the invention, there is provided a method for for the treatment of obesity and its associated complications in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:
a) a compound of formula I, or a pharmaceutically acceptable salt thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:
a) a compound of formula I, or a pharmaceutically acceptable salt thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the the treatment of obesity and its associated complications in a warm-blooded animal, such as man. According to another feature of the invention there is provided the use of a compound of the formula I, or a pharmaceutically acceptable salt thereof, and one of the other compounds is described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man According to a further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatorheic hepatitis, osteoarthritis and some cancers) and psychiatric and neurological conditions.

### EXAMPLES

The invention will now be described in more detail with the following examples that are not to be construed as limiting the invention.

### ABBREVIATIONS

DCM—dichloromethane
DMF—dimethylformamide
DMAP—4-dimethylaminopyridine
EDC—1-(3-dimethylaminopropyl)-3-ethylcarbodiimide
TEA—triethylamine
TEA—trifluoroacetic acid
DMSO dimethyl sulfoxide
t triplet
s singlet
d doublet
q quartet
qvint quintet
m multiplet
br broad
bs broad singlet
dm doublet of multiplet
bt broad triplet
dd doublet of doublets
   General Experimental Procedures

Mass spectra were recorded on either a Micromass ZQ single quadrupole or a Micromass LCZ single quadrupole mass spectrometer both equipped with a pneumatically assisted electrospray interface (LC-MS). ¹H NMR measurements were performed on a Varian Inova 500, operating at ¹H frequency 500 MHz. Chemical shifts are given in ppm. Purifications were performed on a semipreparative HPLC with a mass triggered fraction collector, Shimadzu QP 8000 single quadrupole mass spectrometer equipped with 19×100 mm C8 column. As the mobile phase, acetonitrile and buffered phase (0.1 M NH₄Ac:acetonitrile 95:5) were used.

Alternatively ¹H NMR and ¹³C NMR measurements were performed on a Varian Mercury 300 or Varian UNITY plus 400, 500 or 600 spectrometers, operating at ¹H frequencies of 300, 400, 500 and 600 MHz, respectively, and at ¹³C frequencies of 75, 100, 125 and 150 MHz, respectively. Measurements were made on the delta scale (δ).

Unless otherwise stated, chemical shifts are given in ppm with the solvent as internal standard.

Synthesis of Intermediates

Preparation A

The following intermediates were prepared according to Scalzo, M. et al., Farmaco, Ed. Sci. (1988), 43(9), 665-676.

(a) Ethyl 2-acetyl-4-oxo-4-phenylbutanoate

¹H-NMR ((CD₃)₂SO) δ 7.98 (d, 2H), 7.65 (t, 1H), 7.53 (t, 2H), 4.13 (m, 3H), 3.56 (ddd, 2H), 2.32 (s, 3H), 1.18 (t, 3H).

(b) Ethyl 2-acetyl-4-(2,4-dichlorophenyl)-4-oxobutanoate

¹H-NMR ((CD₃)₂SO) δ 7.81-7.54 (m, 3H), 4.20-4.10 (m, 3H), 3.52-3.39 (m, 2H), 2.30 (s, 3H), 1.18 (t, 3H).

(c) Ethyl 2-acetyl-4-(2,4-dimethoxyphenyl)-4-oxobutanoate

¹H-NMR ((CD₃)₂SO) δ 7.68 (dd, 1H), 6.67 (s, 1H), 6.61 (m, 1H), 4.10 (m, 3H), 3.91, (d, 3H), 3.84 (d, 3H), 3.41 (m, 2H), 2.28 (d, 3H), 1.17 (dt, 3H). MS m/z 309 (M+H)⁺.

Preparation B

The following intermediates were prepared essentially as described: Scalzo, M. et al., Farmaco, Ed. Sci. (1988), 43(9), 665-676. As recognised by those skilled in the art, the compounds described in Preparation A were, together with the appropriately substituted aniline, used as starting materials.

(a) Ethyl 2-methyl-1,5-diphenyl-1H-pyrrole-3-carboxylate

Toluene-4-sulphonic acid monohydrate (13 mg, 0.075 mmol) was added under nitrogen to a solution of aniline (0.43 mL, 4.7 mmol) and ethyl 2-acetyl-4-oxo-4-phenylbutanoate (Preparation A (a), 1.16 g, 4.7 mmol) in ethanol (55 mL). The mixture was refluxed for 20 h, then evaporated. The crude product (1.22 g) was used in the next step without further purification. MS m/z 306 (M+H)⁺.

(b) Ethyl 1-(4-chlorophenyl)-2-methyl-5-phenyl-1H-pyrrole-3-carboxylate

The title compound was prepared as described in Preparation B (a).

The crude product (1.61 g) was used in the next step without further purification. MS m/z 340 (M+H)⁺.

(c) Ethyl 1-(4-methoxyphenyl)-2-methyl-5-phenyl-1H-pyrrole-3-carboxylate

The title compound was prepared as described in Preparation B (a).

The crude product (1.68 g) was used in the next step without further purification MS m/z 336 (M+H)⁺.

(d) Ethyl 5-(2,4-dichlorophenyl)-2-methyl-1-phenyl-1H-pyrrole-3-carboxylate

The title compound was prepared as described in Preparation B (a).

The crude product (0.55 g) was used in the next step without further purification. MS m/z 374 (M+H)⁺.

(e) Ethyl 1-(4-chlorophenyl)-5-(2,4-dichlorophenyl)-2-methyl-1H-pyrrole-3-carboxylate

The title compound was prepared as described in Preparation B (a).

The crude product (1.32 g) was used in the next step without further purification. MS m/z 408 (M+H)⁺.

(f) Ethyl 5-(2,4-dichlorophenyl)-1-(4-methoxyphenyl)-2-methyl-1H-pyrrole-3-carboxylate

The title compound was prepared as described in Preparation B (a).

The crude product (0.72 g) was used in the next step without farther purification. MS m/z 404 (M+H)⁺.

(g) Ethyl 5-(2,4-dimethoxyphenyl)-2-methyl-1-phenyl-1H-pyrrole-3-carboxylate

The title compound was prepared as described in Preparation B (a).

The crude product (0.33 g) was used in the next step without further purification. MS m/z 366 (M+H)⁺.

(h) Ethyl 1-(4-chlorophenyl)-5-(2,4-dimethoxyphenyl)-2-methyl-1H-pyrrole-3-carboxylate

The title compound was prepared as described in Preparation B (a).

The crude product (0.36 g) was used in the next step without further purification. MS m/z 400 (M+H)⁺.

(i) Ethyl 5-(2,4-dimethoxyphenyl)-1-(4-methoxyphenyl)-2-methyl-1H-pyrrole-3-carboxylate

The title compound was prepared as described in Preparation B (a).

The crude product (0.37 g) was used in the next step without further purification. MS m/z 396 (M+H)⁺.

Preparation C

The title compounds described in Preparation B (a-i) were used as starting materials for the compounds described in Preparation C (a-i)

(a) 2-Methyl-1,5-diphenyl-1H-pyrrole-3-carboxylic acid

Sodium hydroxide (2.4 g, 60 mmol) was added to a solution of crude ethyl 2-methyl-1,5-diphenyl-1H-pyrrole-3-carboxylate (from Preparation B (a), 1.22 g, 4.0 mmol) in ethanol (25 mL). The mixture was refluxed for 3 h, then an additional portion of sodium hydroxide (0.20 g, 5.0 mmol) was added and the mixture was refluxed for an additional 90 min. The ethanol was evaporated, then HCl (75 mL, 2M aq) was added and the mixture was stirred for 7 h. The acidic aqueous solution was extracted with EtOAc, the organic layer was washed with brine, dried (MgSO₄), filtrated and concentrated to give the crude product (0.95 g). The crude product was used in the next step without further purification. MS m/z 278 (M+H)⁺.

(b) 1-(4-Chlorophenyl)-2-methyl-5-phenyl-1H-pyrrole-3-carboxylic acid

The title compound was prepared as described in Preparation C (a).

The crude product (1.2 g) was used in the next step without further purification. MS m/z 312 (M+H)⁺.

(c) 1-(4-Methoxyphenyl)-2-methyl-5-phenyl-1H-pyrrole-3-carboxylic acid

The title compound was prepared as described in Preparation C (a).

The crude product (1.3 g) was used in the next step without further purification. MS m/z 308 (M+H)⁺.

(d) 5-(2,4-Dichlorophenyl)-2-methyl-1-phenyl-1H-pyrrole-3-carboxylic acid

The title compound was prepared as described in Preparation C (a).

The crude product (0.44 g) was used in the next step without further purification. MS m/z 346 (M+H)⁺.

(e) 1-(4-Chlorophenyl)-5-(2,4-dichlorophenyl)-2-methyl-1H-pyrrole-3-carboxylic acid

The title compound was prepared as described in Preparation C (a).

The crude product (1.12 g) was used in the next step without further purification. MS m/z 380 (M+H)⁺.

(f) 5-(2,4-Dichlorophenyl)-1-(4-methoxyphenyl)-2-methyl-1H-pyrrole-3-carboxylic acid

The title compound was prepared as described in Preparation C (a).

The crude product (0.51 g) was used in the next step without further purification. MS m/z 376 (M+H)⁺.

(g) 5-(2,4-Dimethoxyphenyl)-2-methyl-1-phenyl-1H-pyrrole-3-carboxylic acid

The title compound was prepared as described in Preparation C (a).

The crude product (0.26 g) was used in the next step without further purification. MS m/z 338 (M+H)⁺.

(h) 1-(4-Chlorophenyl)-5-(2,4-dimethoxyphenyl)-2-methyl-1H-pyrrole-3-carboxylic acid

The title compound was prepared as described in Preparation C (a).

The crude product (0.30 g) was used in the next step without further purification. MS m/z 372 (M+H)⁺.

### (i) 5-(2,4-Dimethoxyphenyl)-1-(4-methoxyphenyl)-2-methyl-1H-pyrrole-3-carboxylic acid

The title compound was prepared as described in Preparation C (a).

The crude product (0.34 g) was used in the next step without further purification. MS m/z 368 (M+H)⁺.

### Examples of the Invention

### Example 1

### 2-Methyl-N,1,5-triphenyl-1H-pyrrole-3-carboxamide

The crude 2-methyl-1,5-diphenyl-1H-pyrrole-3-carboxylic acid ( 50 mg, 0.18 mmol) from Preparation C (a) and 4-dimethylaminopyridine (10 mg, 0.08 mmol) were dissolved in CH₂Cl₂ (2 mL) and DMF (0.030 mL). The solution was cooled to 0° C. A slurry of 1-ethyl-3-(3-dimethylaminopropyl)carbodiimide hydrochloride (76 mg, 0.40 mmol) in CH₂Cl₂ (0.5 mL) and DMF (0.040 mL) was added dropwise. Aniline (0.046 mL, 0.49 mmol) in CH₂Cl₂ (0.5 mL) and was then added dropwise. The mixture was allowed to attain room temperature, and was stirred overnight. The mixture was diluted with CH₂Cl₂, washed with Na₂HCO₃ (sat, aq) and the phases were separated. The organic phase was concentrated and the residue was purified by semipreparative HPLC to give the title compound (33 mg, 52%).

¹H-NMR (CD₃OD) δ 7.65 (dd, 2H), 7.44 (m, 3H), 7.33 (t, 2H), 7.20 (m, 2H), 7.16-7.08 (m, 6H), 6.90 (s, 1H), 2.38 (s, 3H). MS m/z 353 (M+H)⁺.

### Example 2

### 1-(4-Chlorophenyl-2-methyl-N,5-diphenyl-1H-pyrrole-3-carboxamide

Crude 1-(4-chlorophenyl)-2-methyl-5-phenyl-1H-pyrrole-3-carboxylic acid from Preparation C (b) was used as described in Example 1 to give the title compound (31 mg, 50%). ¹H-NMR (CD₃OD) δ 7.65 (d, 2H), 7.45 (m, 2H), 7.33 (t, 2H), 7.22-7.08 (m, 8H), 6.90 (s, 1H), 2.40 (s, 3H). MS m/z 387 (M+H)⁺.

### Example 3

### 1-(4-methoxyphenyl)-2-methyl-N,5-diphenyl-1H-pyrrole-3-carboxamide

Crude 1-(4-methoxyphenyl)-2-methyl-5-phenyl-1H-pyrrole-3-carboxylic acid from Preparation C (c) was used as described in Example 1 to give the title compound (20 mg, 32%). ¹H-NMR (CD₃OD) δ 7.65 (d, 2H), 7.33 (t, 2H), 7.18-7.08 (m, 8H), 6.97 (m, 2H), 6.88 (s, 1H), 3.82 (s, 3H), 2.37 (s, 3H). MS m/z 383 (M+H)⁺.

### Example 4

### 5-(2,4-dichlorophenyl)-2-methyl-N,1-diphenyl-1H-pyrrole-3-carboxamide

Crude 5-(2,4-dichlorophenyl)-2-methyl-1-phenyl-1H-pyrrole-3-carboxylic acid from Preparation C (d) was used as described in Example 1 to give the title compound (9 mg, 15%). ¹H-NMR (CD₃OD) δ 7.64 (dd, 2H), 7.39-7.30 (m, 6H), 7.23 (d, 1H), 7.17 (m, 3H), 7.10 (dt, 1H), 6.84 (s, 1H), 2.40 (s, 3H). MS m/z 421 (M+H)⁺.

### Example 5

### 1-(4-Chlorophenyl)-5-(2,4-dichlorophenyl-2-methyl-N-phenyl-1H-pyrrole-3-carboxamide

Crude 1-(4-chlorophenyl)-5-(2,4-dichlorophenyl)-2-methyl-1H-pyrrole-3-carboxylic acid from Preparation C (e) was used as described in Example 1 to give the title compound (3 mg, 5%). ¹H-NMR (CD₃OD) δ 7.64 (dd, 2H), 7.41-7.36 (m, 3H), 7.32 (t, 2H), 7.27 (d, 1H), 7.23 (dd, 1H), 7.17 (m, 2H), 7.10 (t, 1H), 6.85 (s, 1H), 2.42 (s, 3H). MS m/z 455 (M+H)⁺.

### Example 6

### 5-(2,4-Dichlorophenyl)-1-(4methoxyphenyl-2-methyl-N-phenyl-1H-pyrrole-3-carboxamide

Crude 5-(2,4-dichlorophenyl)-1-(4-methoxyphenyl)-2-methyl-1H-pyrrole-3-carboxylic acid from Preparation C (f) was used as described in Example 1 to give the title compound (15 mg, 25%). ¹H-NMR (CD₃OD) δ 7.64 (dd, 2H), 7.38 (d, 1H), 7.32 (t, 2H), 7.22 (t, 1H), 7.19 (dd, 1H), 7.09 (m, 3H), 6.89 (m, 2H), 6.82 (s, 1H), 3.78 (s, 3H), 2.38 (s, 3H). MS m/z 451 (M+H)⁺.

### Example 7

### 5-(2,4-Dimethoxyphenyl)-2-methyl-N,1-diphenyl-1H-pyrrole-3-carboxamide

Crude 5-(2,4-dimethoxyphenyl)-2-methyl-1-phenyl-1H-pyrrole-3-carboxylic acid from Preparation C (g) was used as described in Example 1 to give the title compound (20 mg, 33%). ¹H-NMR (CD₃OD) δ 7.64 (dd, 2H), 7.36-7.24 (m, 5H), 7.15-7.06 (m, 4H), 6.65(s, 1H), 6.43 (dd, 1H), 6.28 (d, 1H), 3.73 (s, 3H), 3.42 (s, 3H), 2.38 (s, 3H). MS m/z 413 (M+H)⁺.

### Example 8

### 1-(4-Chlorophenyl)-5-(2,4-dimethoxyphenyl)-2-methyl-N-phenyl-1H-pyrrole-3-carboxamide

Crude 1-(4-chlorophenyl)-5-(2,4-dimethoxyphenyl)-2-methyl-1H-pyrrole-3-carboxylic acid from Preparation C (h) was used as described in Example 1 to give the title compound (39 mg, 65%). ¹H-NMR (CD₃OD) δ 7.63 (d, 2H), 7.32 (m, 4H), 7.17-7.06 (m, 4H), 6.65(s, 1H), 6.46 (dd, 1H), 6.31 (d, 1H), 3.75 (s, 3H), 3.44 (s, 3H), 2.39 (s, 3H). MS m/z 447 (M+H)⁺.

### Example 9

### 5-(2,4-Dimethoxyphenyl)-1-(4-methoxyphenyl)-2-methyl-N-phenyl-1H-pyrrole-3-carboxamide

Crude 5-(2,4-dimethoxyphenyl)-1-(4-methoxyphenyl)-2-methyl-1H-pyrrole-3-carboxylic acid from Preparation C (i) was used as described in Example 1 to give the title compound (44 mg, 73%). ¹H-NMR (CD₃OD) δ 7.63 (d, 2H), 7.32 (t, 2H), 7.09 (m, 2H), 7.00 (d, 2H), 6.85 (d, 2H), 6.62(s, 1H), 6.42 (dd, 1H), 6.31 (d, 1H), 3.77 (s, 3H), 3.73 (s, 3H), 3.48 (s, 3H), 2.36 (s, 3H). MS m/z 443 (M+H)⁺.

### Example 10a

### 2-Methyl-1,5-diphenyl-N-piperidin-1-yl-1H-pyrrole-3-carboxamide

### And Example 10b

### 1-[(2-Methyl-1,5-diphenyl-1H-pyrrol-3-yl)carbonyl]piperidine

The crude 2-methyl-1,5-diphenyl-1H-pyrrole-3-carboxylic acid (236 mg, 0.85 mmol) from Preparation C (a) and 4-dimethylaminopyridine (47 mg, 0.38 mmol) were dissolved in CH₂Cl₂ (5 mL) and DMF (0.142 mL) and 1-aminopiperidine (0.218 mL, 2.18 mmol) was added. The solution was cooled to 0° C. A slurry of 1-ethyl-3-(3-dimethylaminopropyl)-carbodiimide hydrochloride (360 mg, 01.88 mmol) in CH₂Cl₂ (2.4 nm) and DMF (0.189 mL) was added dropwise. The mixture was allowed to attain room temperature, and was stirred overnight. The mixture was diluted with CH₂Cl₂, washed with Na₂HCO₃ (sat, aq) and the phases were separated. The organic phase was concentrated and the residue was purified by semipreparative HPLC to give 10a (20 mg, 7%), and 10b (91 mg, 31%).

10a had: ¹H-NMR (CD₃OD) δ 7.41 (m, 3H), 7.20-7.04 (m, 71H), 6.68 (s, 1H), 2.84 (brs, 4H), 2.32 (s, 3H), 1.74 (m, 4H), 1.46 (brs, 2H). MS m/z 360 (M+H)⁺.

10b had: ¹H-NMR (CD₃OD) δ 7.41 (m, 3H), 7.20-7.04 (m, 7H), 6.37 (s, 1H), 3.70 (t, 4H), 2.32 (s, 3H), 1.74 (m, 2H), 1.65 (brs, 4H). MS m/z 345 (M+H)⁺.

### Example 11a

### 1-(4-Chlorophenyl)-2-methyl-5-phenyl-N-piperidin-1-yl-1H-pyrrole-3-carboxamide

### And Example 11b

### 1-{[1-(4-Chlorophenyl)-2-methyl-5-phenyl-1H-pyrrol-3-yl]carbonyl}piperidine

Crude 1-(4-chlorophenyl)-2-methyl-5-phenyl-1H-pyrrole-3-carboxylic acid from Preparation C (b) was used as described in Example 10 to give the title compounds 11a (7 mg, 2%), and 11b (129 mg, 35%).

11a had: ¹H-NMR (CD₃OD) δ 7.43 (m, 2H), 7.20-7.04 (m, 7H), 6.67 (s, 1H), 2.83 (brs, 4H), 2.34 (s, 3H), 1.74 (m, 4H), 1.46 (brs, 2H). MS m/z 394 (M+H)⁺.

11b had: ¹H-NMR (CD₃OD) δ 7.43 (m, 2H), 7.20-7.04 (m, 7H), 6.37 (s, 1H), 3.68 (t, 4H), 2.12 (s, 3H), 1.74 (m, 2H), 1.64 (brs, 4H). MS m/z 379 (M+H1)⁺.

### Example 12a

### 1-(4-Methoxyphenyl)-2-methyl-5-phenyl-N-piperidin-1-yl-1H-pyrrole-3-carboxamide

### And Example 12b

### 1-{[1-(4-Methoxyphenyl)-2-methyl-5-phenyl-1H-pyrrol-3-yl]carbonyl}piperidine

Crude 1-(4-methoxyphenyl)-2-methyl-5-phenyl-1H-pyrrole-3-carboxylic acid from Preparation C (c) was used as described in Example 10 to give the title compounds 12a (43 mg, 10%), and 12b (174 mg, 43%).

12a had: ¹H-NMR (CD₃OD) δ 7.16-7.05 (m, 7H), 6.96 (d, 2H), 6.66 (s, 1H), 3.81 (s, 3H), 2.83 (brs, 4H), 2.50 (s, 3H), 1.74 (m, 4H), 1.45 (brs, 2H). MS m/z 390 (M+H)⁺.

12b had: ¹H-NMR (CD₃OD) δ 7.16-7.05 (m, 7H), 6.95 (d, 2H), 6.35 (s, 1H), 3.81 (s, 3H), 3.70 (brs, 4H), 2.10 (s, 3H), 1.74 (m, 2H), 1.64 (brs, 4H). MS m/z 375 (M+H)⁺.

### Example 13a

### 5-(2,4-Dichlorophenyl)-2-methyl-1-phenyl-N-piperidin-1-yl-1H-pyrrole-3-carboxamide

### And Example 13b

### 1-{[5-(2,4-Dichlorophenyl)-2-methyl-1-phenyl-1H-pyrrol-3-yl]carbonyl}piperidine

Crude 5-(2,4-dichlorophenyl)-2-methyl-1-phenyl-1H-pyrrole-3-carboxylic acid from Preparation C (d) was used as described in Example 10 to give the title compounds 13a (7 mg, 3%), and 13b (52 mg, 20%).

13a had: ¹H-NMR (CD₃OD) δ 7.37-7.30 (m, 4H), 7.20-7.10 (m, 4H), 6.61 (s, 1H), 2.82 (brs, 4H), 2.35 (s, 3H), 1.73 (t, 4H), 1.45 (brs, 2H). MS m/z 428 (M+H)⁺.

13b had: ¹H-NMR (CD₃OD) δ 7.38-7.30 (m, 4H), 7.15 (m, 4H), 6.34 (s, 1H), 3.70 (t, 4H), 2.15 (s, 3H), 1.75 (t, 2H), 1.64 (brs, 4H). MS m/z 413 (M+H)⁺.

### Example 14a

### 1-(4-Chlorophenyl)-5-(2,4-dichlorophenyl)-2-methyl-N-piperidin-1-yl-1H-pyrrole-3-carboxamide

### And Example 14b

### 1-{[1-(4-Chlorophenyl)-5-(2,4-dichlorophenyl)-2-methyl-1H-pyrrol-3-yl]carbonyl}piperidine

Crude 1-(4-chlorophenyl)-5-(2,4-dichlorophenyl)-2-methyl-1H-pyrrole-3-carboxylic acid from Preparation C (e) was used as described in Example 10 to give the title compounds 14a (17 mg, 3%), and 14b (144 mg, 22%).

14a had: ¹H-NMR (CD₃OD) δ 7.36 (m 3H), 7.22 (s, 2H), 7.13 (m, 2H), 6.62 (s, 1H), 2.80 (brs, 4H), 2.35 (s, 3H), 1.72 (t, 4H), 1.44 (brs, 2H). MS m/z 462 (M+H)⁺.

14b had: ¹H-NMR (CD₃OD) δ 7.37 (m, 3H), 7.20 (s, 2H), 7.15 (d, 2H), 6.34 (s, 1H), 3.69 (t, 4H), 2.15 (s, 3H), 1.73 (m, 2H), 1.62 (brs, 4H). MS m/z 447 (M+H)⁺.

### Example 15a

### 5-(2,4-Dichlorophenyl-1-(4-methoxyphenyl)-2-methyl-N-piperidin-1-yl-1H-pyrrole-3-carboxamide

### And Example 15b

### 1-{[5-(2,4-Dichlorophenyl)-1-(4-methoxyphenyl)-2-methyl-1H-pyrrol-3-yl]carbonyl}piperidine

Crude 5-(2,4-dichlorophenyl)-1-(4-methoxyphenyl)-2-methyl-1H-pyrrole-3-carboxylic acid from Preparation C (f) was used as described in Example 10 to give the title compounds 15a (24 mg, 8%), and 15b (69 mg, 23%).

15a had: ¹H-NMR (CD₃OD) δ 7.36 (s, 1H), 7.17 (s, 2H), 7.04 (d, 2H), 6.87 (d, 2H), 6.58 (s, 1H), 3.76 (s, 3H), 2.82 (brs, 4H), 2.37 (s, 3H), 1.72 (m, 4H), 1.44 (brs, 2H). MS m/z 458 (M+H)⁺.

15b had: ¹H-NMR (CD₃OD) δ 7.37 (s, 1H), 7.15 (s, 2H), 7.06 (m, 2H), 6.88 (m, 2H), 6.31 (s, 1H), 3.77 (s, 3H), 3.69 (t, 4H), 2.13 (s, 3H), 1.73 (m, 2H), 1.62 (brs, 4H). MS m/z 443 (M+H)⁺.

### Example 16

### 1-{[5-(2,4-Dimethoxyphenyl)-2-methyl-1-phenyl-1H-pyrrol-3-yl]carbonyl}piperidine

Crude 5-(2,4-dimethoxyphenyl)-2-methyl-1-phenyl-1H-pyrrole-3-carboxylic acid from Preparation C (g) was used as described in Example 10 to give the title compound (83 mg, 54%).

¹H-NMR (CD₃OD) δ 7.34-7.20 (m, 3H), 7.07 (m, 3H), 6.40 (m, 1H), 6.27 (s, 1H), 6.15 (s, 1H), 3.70 (m, 7H), 3.39 (s, 3H), 2.14 (s, 3H), 1.73 (m, 2H), 1.63 (brs, 4H). MS m/z 405 (M+H)⁺.

### Example 17a

### 1-(4-Chlorophenyl)-5-(2,4-dimethoxyphenyl)-2-methyl-N-piperidin-1-yl-1H-pyrrole-3-carboxamide

### And Example 17b

### 1-{[1-(4Chlorophenyl)-5-(2,4-dimethoxyphenyl)-2-methyl-1H-pyrrol-3-yl]carbonyl}piperidine

Crude 1-(4-chlorophenyl)-5-(2,4-dimethoxyphenyl)-2-methyl-1H-pyrrole-3-carboxylic acid from Preparation C (h) was used as described in Example 10 to give the title compounds 17a (4 mg, 7%) and 17b (47 mg, 27%).

¹H-NMR (CD₃OD) for 17a: δ 7.31 (d, 2H), 7.07 (m, 3H), 6.43 (m, 2H), 6.30 (s, 1H), 3.74 (s, 3H), 3.41 (s, 3H), 2.80 (brs, 4H), 2.33 (s, 3H), 1.72 (m, 4H), 1.44 (brs, 2H). MS m/z 454 (M+H)⁺.

¹H-NMR (CD₃OD) for 17b: δ 7.32 (d, 2H), 7.07 (m, 3H), 6.44 (m, 1H), 6.30 (s, 1H), 6.15 (s, 1H), 3.74 (s, 3H), 3.69 (m, 4H), 3.41 (s, 3H), 2.14 (s, 3H), 1.72 (m, 2H), 1.62 (brs, 4H). MS m/z 439 (M+H)⁺.

### Example 18a

### 5-(2,4-Dimethoxyphenyl)-1-(4-methoxyphenyl)-2-methyl-N-piperidin-1-yl-1H-pyrrole-3-carboxamide

### And Example 18b

### 1-{[5-(2,4-Dimethoxyphenyl)-1-(4-methoxyphenyl)-2-methyl-1H-pyrrol-3-yl]carbonyl}piperidine

Crude 5-(2,4-dimethoxyphenyl)-1-(4-methoxyphenyl)-2-methyl-1H-pyrrole-3-carboxylic acid from Preparation C (i) was used as described in Example 10 to give the title compounds 18a (45 mg, 22%), and 18b (92 mg, 56%).

18a had: ¹H-NMR (CD₃OD) δ 7.04 (d, 1H), 6.97 (m, 2H), 6.84 (m, 2H), 6.40 (m, 2H), 6.29 (d, 1H), 3.76 (s, 3H), 3.74 (s, 3H), 3.48 (s, 3H), 2.82 (brs, 4H), 2.40 (s, 3H), 1.72 (m, 4H), 1.44 (brs, 2H). MS m/z 450 (M+H)⁺.

18b had: ¹H-NMR (CD₃OD) δ 7.03 (d, 1H), 6.98 (m, 2H), 6.84 (m, 2H), 6.40 (dd, 1H), 6.30 (d, 1H), 6.11 (s, 1H), 3.75 (s, 3H), 3.72 (s, 3H), 3.69 (brs, 4H), 3.46 (s, 3H), 2.11 (s, 3H), 1.73 (m, 2H), 1.62 (brs, 4H). MS m/z 435 (M+H)⁺.

Pharmacological Activity

Compounds of the present invention are active against the receptor product of the CB1 gene. The affinity of the compounds of the invention for central cannabinoid receptors is demonstrable in methods described in Devane et al, Molecular Pharmacology, 1988, 34,605 or those described in WO01/70700 or EP 656354. Alternatively the assay may be performed as follows.

10 μg of membranes prepared from cells stably transfected with the CB1 gene were suspended in 200 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x)ÚD)) and the IC50 value determined as the concentration required to give half maximal inhibition of GTPγS binding under the conditions used.

The compounds of the present invention are active at the CB1 receptor (IC50<1 micromolar). Most preferred compounds have IC50<200 nanomolar.

1. A compound of formula (I) 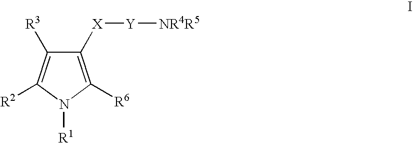
wherein
R¹ and R² are independently selected from phenyl, thienyl and pyridyl, each of which is independently optionally substituted with one, two or three Z groups;
Z is selected from a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, amino, mono or di C₁₋₃alkylamino, mono or di C₁₋₃alkylamido, C₁₋₃alkylsulphonyl, C₁₋₃alkoxycarbonyl, carboxy, cyano, carbamoyl, mono or di C₁₋₃alkyl carbamoyl, sulphamoyl and acetyl;
R³ is selected from H, a C₁₋₃alkyl group, a C₁₋₃alkoxymethyl group, trifluoromethyl, an aminoC₁₋₃alkyl group, a hydroxyC₁₋₃alkyl group, C₁₋₃alkoxycarbonyl, carboxy, cyano, carbamoyl, mono or di C₁₋₃alkylcarbamoyl, acetyl, and —CONHNR^{a}R^{b}, wherein R^{a} and R^{b} are R⁴ and R⁵, respectively; and
X is CO or SO₂;
Y is absent or NH, optionally substituted with a C₁₋₃alkyl group;
R⁴ and R⁵ are independently selected from:
a C₁₋₆alkyl group;
an (amino)C₁₋₄alkyl-group in which the amino is optionally substituted with one or more C₁₋₃alkyl groups;
an optionally substituted non-aromatic C₃₋₁₅carbocyclic group;
a (C₃₋₁₂cycloalkyl)C₁₋₃alkyl-group;
a —(CH₂)ᵣ(phenyl)ₛ group, wherein r is 0, 1, 2, 3 or 4, and wherein s is 1 when r is 0, otherwise s is 1 or 2, and wherein the phenyl groups are optionally independently substituted with one, two or three Z groups represented;
naphthyl;
anthracenyl;
a saturated 5- to 8-membered heterocyclic group containing one nitrogen and optionally one of the following: oxygen, sulphur or an additional nitrogen, wherein the heterocyclic group is optionally substituted with one or more C₁₋₃alkyl groups, hydroxy or benzyl;
1-adamantylmethyl; and
a —(CH₂)ₜHet group, wherein t is 0, 1, 2, 3 or 4, and the alkylene chain is optionally substituted with one or more C₁₋₃alkyl groups and wherein Het is an aromatic heterocycle optionally substituted with one, two or three groups selected from a C₁₋₅alkyl group, a C₁₋₅alkoxy group and halo;
or R⁴ is H and R⁵ is as defined above;
or R⁴ and R⁵ taken together with the nitrogen atom to which they are attached form a saturated 5- to 8-membered heterocyclic group containing one nitrogen and optionally one of the following: oxygen, sulphur or an additional nitrogen; wherein the heterocyclic group is optionally substituted with one or more C₁₋₃alkyl groups, hydroxy or benzyl;
R⁶ is selected from H, a C₁₋₃alkyl group, a C₁₋₃alkoxymethyl group, trifluoromethyl, a hydroxyC₁₋₃alkyl group, C₁₋₃alkoxycarbonyl, carboxy, cyano, carbamoyl, mono or di C₁₋₃alkylcarbamoyl, acetyl, and —CONHNR^{a}R^{b}, wherein R^{a} and R^{b} are R⁴ and R⁵, respectively; and
with the proviso that when R⁶ is methyl, then the group X—Y—NR⁴R⁵ is not CONHC₆H₁₃, CONHC₁₂H₂₅, CONH₂, CONHCH₃, CON(CH₃)₂, 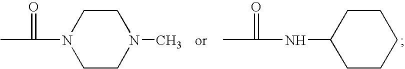
and with the further proviso that when R¹ and R² are independently phenyl, then Z is not an ortho methyl group;
or a pharmaceutically acceptable salt, prodrug or solvate thereof.

2. A compound according to claim 1, wherein R¹ is phenyl optionally substituted in the 2 or 4 position with halo or C₁₋₃alkoxy.

3. A compound according to claim 1, wherein R² is phenyl, optionally substituted in the 2 or 4 position with halo or C₁₋₃alkoxy.

4. A compound according to claim 1, wherein X—Y—NR⁴R⁵ is CONHPh or CONH(1-piperidyl).

5. A compound according to claim 1, wherein R⁶ is methyl.

6. A compound according to claim 1 of the general formula (II) 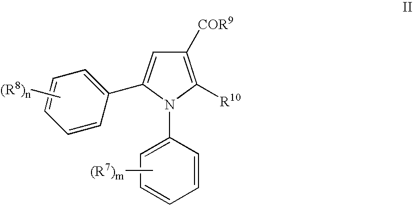
wherein
m is 0, 1, 2 or 3;
each R⁷ is independently selected from a C₁₋₆alkyl group, trifluoromethyl, a C₁₋₆alkoxy group, difluoromethoxy, trifluoromethoxy, and halo;
n is 0,1, 2 or 3;
each R⁸ is indepently selected from a C₁₋₆alkyl group, trifluoromethyl, a C₁₋₆alkoxy group, difluoromethoxy, trifluoromethoxy, and halo;
R⁹ is selected from 1-piperidinyl, 1-piperidinylamino and anilino, wherein the phenyl ring is optionally substituted with one or more of the following: a C₁₋₆alkyl group, trifluoromethyl, a C₁₋₆alkoxy group, difluoromethoxy, trifluoromethoxy, or halo; and
R¹⁰ is selected from a C₁₋₆alkyl, C₁₋₆alkoxy, and a C₁₋₆alkylamino group;
or a pharmaceutically acceptable salt, prodrug or solvate thereof;
with the proviso that the compound is not 1-{[1-(4-chlorophenyl)-5-phenyl-2-methyl-1H-pyrrol-3-yl]carbonyl}piperidine or 1-{[1-(2,4-dichlorophenyl)-5-phenyl-2-methyl-1H-pyrrol-3-yl]carbonyl}piperidine.

7. A compound according to claim 6, wherein m is 2 and each R⁷, if present, is located in the 2 or 4 position of the phenyl ring.

8. A compound according to claim 6, wherein n is 2 and each R⁸, if present, is located in the 2 or 4 position of the phenyl ring.

9. A compound according to claim 6, wherein R⁹ is 1-piperidinyl.

10. A compound according to claim 6, wherein R⁹ is 1-piperidinylamino.

11. A compound according to claim 6, wherein R¹⁰ is methyl.

12. A compound selected from:
2-methyl-N,1,5-triphenyl-1H-pyrrole-3-carboxamide;
1-(4-chlorophenyl)-2-methyl-N,5-diphenyl-1H-pyrrole-3-carboxamide;
1-(4-methoxyphenyl)-2-methyl-N,5-diphenyl-1H-pyrrole-3-carboxamide;
5-(2,4-dichlorophenyl)-2-methyl-N,1-diphenyl-1H-pyrrole-3-carboxamide;
1-(4-chlorophenyl)-5-(2,4-dichlorophenyl)-2-methyl-N-phenyl-1H-pyrrole-3-carboxamide;
5-(2,4-dichlorophenyl)-1-(4-methoxyphenyl)-2-methyl-N-phenyl-1H-pyrrole-3-carboxamide;
5-(2,4-dimethoxyphenyl)-2-methyl-N,1-diphenyl-1H-pyrrole-3-carboxamide;
1-(4-chlorophenyl)-5-(2,4-dimethoxyphenyl)-2-methyl-N-phenyl-1H-pyrrole-3-carboxamide;
5-(2,4-dimethoxyphenyl)-1-(4-methoxyphenyl)-2-methyl-N-phenyl-1H-pyrrole-3-carboxamide;
2-methyl-1,5-diphenyl-N-piperidin-1-yl-1H-pyrrole-3-carboxamide;
1-(4-chlorophenyl)-2-methyl-5-phenyl-N-piperidin-1-yl-1H-pyrrole-3-carboxamide;
1-(4-methoxyphenyl)-2-methyl-5-phenyl-N-piperidin-1-yl-1H-pyrrole-3-carboxamide;
5-(2,4-dichlorophenyl)-2-methyl-1-phenyl-N-piperidin-1-yl-1H-pyrrole-3-carboxamide;
1-(4-chlorophenyl)-5-(2,4-dichlorophenyl)-2-methyl-N-piperidin-1-yl-1H-pyrrole-3-carboxamide;
5-(2,4-dichlorophenyl)-1-(4-methoxyphenyl)-2-methyl-N-piperidin-1-yl-1H-pyrrole-3-carboxamide;
1-{[5-(2,4-dimethoxyphenyl)-2-methyl-1-phenyl-1H-pyrrol-3-yl]carbonyl}piperidine;
1-(4-chlorophenyl)-5-(2,4-dimethoxyphenyl)-2-methyl-N-piperidin-1-yl-1H-pyrrole-3-carboxamide;
5-(2,4-dimethoxyphenyl)-1-(4-methoxyphenyl)-2-methyl-N-piperidin-1-yl-1H-pyrrole-3-carboxamide;
1-[(2-methyl-1,5-diphenyl-1H-pyrrol-3-yl)carbonyl]piperidine;
1-{[1-(4-methoxyphenyl)-2-methyl-5-phenyl-1H-pyrrol-3-yl]carbonyl}piperidine;
1-{[5-(2,4-dichlorophenyl)-2-methyl-1-phenyl-1H-pyrrol-3-yl]carbonyl}piperidine;
1-{[1-(4-chlorophenyl)-5-(2,4-dichlorophenyl)-2-methyl-1H-pyrrol-3-yl]carbonyl}piperidine;
1-{[5-(2,4-dichlorophenyl)-1-(4-methoxyphenyl)-2-methyl-1H-pyrrol-3-yl]carbonyl}piperidine;
1-{[1-(4-chlorophenyl)-5-(2,4-dimethoxyphenyl)-2-methyl-1H-pyrrol-3-yl]carbonyl}piperidine; and
1-{[5-(2,4-dimethoxyphenyl)-1-(4-methoxyphenyl)-2-methyl-1H-pyrrol-3-yl]carbonyl}piperidine;
and where applicable, optical isomers, tautomers, stereoisomers and racemates thereof as well as pharmaceutically acceptable salts and solvates thereof.

13. (canceled)

14. A pharmaceutical composition comprising a compound of any one of claims 1 to 12 and a pharmaceutically acceptable adjuvant, diluent or carrier.

15. (canceled)

16. A method of treating a condition selected from obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy, neurological disorders, dementia, neurological disorders, Parkinson's Disease, Huntington's Chorea and Alzheimer's Disease, immune, cardiovascular, reproductive and endocrine disorders, septic shock, diseases related to the respiratory and gastrointestinal systems, and extended abuse, addiction and/or relapse indications, in a mammal, comprising administering a pharmacologically effective amount of a compound of formula (I) 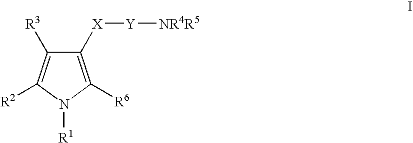
wherein
R¹ and R² are independently selected from phenyl, thienyl and pyridyl, each of which is independently optionally substituted with one, two or three Z groups,
Z is selected from a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, amino, mono or di C₁₋₃alkylamino, mono or di C₁₋₃alkylamido, C₁₋₃alkylsulphonyl, C₁₋₃alkoxycarbonyl, carboxy, cyano, carbamoyl, mono or di C₁₋₃alkyl carbamoyl, sulphamoyl and acetyl;
R³ is selected from H, a C₁₋₃alkyl group, a C₁₋₃alkoxymethyl group, trifluoromethyl, an aminoC₁₋₃alkyl group, a hydroxyC₁₋₃alkyl group, C₁₋₃alkoxycarbonyl, carboxy, cyano, carbamoyl, mono or di C₁₋₃alkylcarbamoyl, acetyl, and —CONHNR^{a}R^{b}, wherein R^{a} and R^{b} are R⁴ and R⁵, respectively: and
X is CO or SO₂;
Y is absent or NH, optionally substituted with a C₁₋₃alkyl group:
R⁴ and R⁵ are independently selected from:
a C₁₋₆alkyl group;
an (amino)C₁₋₄alkyl-group in which the amino is optionally substituted with one or more C₁₋₃alkyl groups:
an optionally substituted non-aromatic C₃₋₁₅ carbocyclic group;
a (C₃₋₁₂cycloalkyl)C₁₋₃al alkyl-group;
a —(CH₂)ᵣ(phenyl)ₛ group, wherein r is 0, 1, 2, 3 or 4, and wherein s is 1 when r is 0, otherwise s is 1 or 2, and wherein the phenyl groups are optionally independently substituted with one, two or three Z groups:
naphthyl:
anthracenyl
a saturated 5- to 8-membered heterocyclic group containing one nitrogen and optionally one of the following: oxygen, sulphur or an additional nitrogen, wherein the heterocyclic group is optionally substituted with one or more C₁₋₃alkyl groups, hydroxy or benzyl:
1-adamantylmethyl; and
a —(CH₂)ₜHet group, wherein t is 0, 1, 2, 3 or 4, and the alkylene chain is optionally substituted with one or more C₁₋₃alkyl groups and wherein Het is an aromatic heterocycle optionally substituted with one, two or three groups selected from a C₁₋₅alkyl group, a C₁₋₅alkoxy group and halo;
or R⁴ is H and R⁵ is as defined above:
or R⁴ and R⁵ taken together with the nitrogen atom to which they are attached form a saturated 5- to 8-membered heterocyclic group containing one nitrogen and optionally one of the following: oxygen, sulphur or an additional nitrogen; wherein the heterocyclic group is optionally substituted with one or more C₁₋₃alkyl groups, hydroxy or benzyl; and
R⁶ is selected from H, a C₁₋₃alkyl group, a C₁₋₃alkoxymethyl group, trifluoromethyl, a hydroxyC₁₋₃alkyl group, C₁₋₃alkoxycarbonyl, carboxy, cyano, carbamoyl, mono or di C₁₋₃alkylcarbamoyl acetyl, and —CONHNR^{a}R^{b}, wherein R^{a} and R^{b} are R⁴ and R⁵, respectively.
to a patient in need thereof.

17. (canceled)

18. A process for the preparation of a compounds of claim 1 in which X is CO,
comprising reacting a compound of formula III 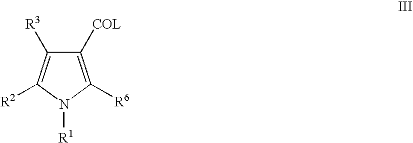
in which R¹, R², R³, and R⁶ are as previously defined and wherein L is hydroxy or halo, with an amine of formula IV
R⁴R⁵YNH₂ IV
in which R⁴ and R⁵ are as previously defined, in an inert solvent and optionally in the presence of a catalyst or optionally in the presence of a base at a temperature in the range of −25° C. to 150° C., and, when L is hydroxy, optionally in the presence of a coupling agent.

19. A compound of formula III 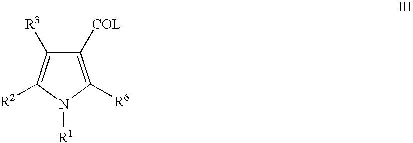
wherein R¹, R², R³, and R⁶ are as defined in claim 1 and L is hydroxy or halo.

20. A compound selected from one or more of the following:
Ethyl 2-methyl-1,5-diphenyl-1H-pyrrole-3-carboxylate,
Ethyl 1-(4-chlorophenyl)-2-methyl-5-phenyl-1H-pyrrole-3-carboxylate,
Ethyl 1-(4-methoxyphenyl)-2-methyl-5-phenyl-1H-pyrrole-3-carboxylate,
Ethyl 5-(2,4-dichlorophenyl)-2-methyl-1-phenyl-1H-pyrrole-3-carboxylate,
Ethyl 1-(4-chlorophenyl)-5-(2,4-dichlorophenyl)-2-methyl-1H-pyrrole-3-carboxylate,
Ethyl 5-(2,4-dichlorophenyl)-1-(4-methoxyphenyl)-2-methyl-1H-pyrrole-3-carboxylate,
Ethyl 5-(2,4-dimethoxyphenyl)-2-methyl-1-phenyl-1H-pyrrole-3-carboxylate,
Ethyl 1-(4-chlorophenyl)-5-(2,4-dimethoxyphenyl)-2-methyl-1H-pyrrole-3-carboxylate,
Ethyl 5-(2,4-dimethoxyphenyl)-1-(4-methoxyphenyl)-2-methyl-1H-pyrrole-3-carboxylate,
2-Methyl-1,5-diphenyl-1H-pyrrole-3-carboxylic acid,
1-(4-Chlorophenyl)-2-methyl-5-phenyl-1H-pyrrole-3-carboxylic acid,
5-(2,4-Dichlorophenyl)-2-methyl-1-phenyl-1H-pyrrole-3-carboxylic acid,
1-(4-Chlorophenyl)-5-(2,4-dichlorophenyl)-2-methyl-1H-pyrrole-3-carboxylic acid,
5-(2,4-Dichlorophenyl)-1-(4-methoxyphenyl)-2-methyl-1H-pyrrole-3-carboxylic acid,
5-(2,4-Dimethoxyphenyl)-2-methyl-1-phenyl-1H-pyrrole-3-carboxylic acid,
1-(4-Chlorophenyl)-5-(2,4-dimethoxyphenyl)-2-methyl-1H-pyrrole-3-carboxylic acid, and
5-(2,4-Dimethoxyphenyl)-1-(4-methoxyphenyl)-2-methyl-1H-pyrrole-3-carboxylic acid.

21. The composition according to claim 14, comprising an additional agent useful in the treatment of hypertension, hyperlipidaemias, dyslipidaemias, diabetes or atherosclerosis.
